(19) United States
(12) Patent Application Publication
(10) Pub. No.: US 2003/0133922 A1
(43) Pub. Date: Jul. 17, 2003

(54) Oral tolerance using allogeneic platelets in ITP

(76) Inventor: John R. Kasha JR., Falls Church, VA (US)

Correspondence Address: John R. Kasha Jr., 7072 Falls Reach Drive, Falls Church, VA, 22034, US

(21) Appl. No.: 10/045,189

(22) Filed: Jan. 15, 2002

(51) Int. Cl. A61K 35/18

(52) U.S. Cl. 424/93.72

(57) ABSTRACT

ITP may be treated using a method that involves identifying the autoimmune response, collecting allogeneic platelets, sterilizing allogeneic platelets and feeding them orally. Auto-antigens contained on the surface of allogeneic platelets administered to the intestines deactivate or delete lymphocytes responsible for auto-antibody production. This treatment can be used for ITP, specifically targets the cause of the disease and provides the possibility of a sustained response without further medication.

### BACKGROUND

1. Field of the Invention

This invention relates to a method of treating Immune Thrombocytopenic Purpura (ITP). In particular, this invention describes a method for treating ITP, which involves identifying the immune response, collecting allogeneic platelets, sterilizing allogeneic platelets and feeding them orally.

2. Description of Prior Art

An autoimmune disease is one in which self tissue is under attack by the body's own immune system. Specifically, in an autoimmune disease antibodies (auto-antibodies) are produced which are directed against and cause the destruction of normal body tissue.

The American Autoimmune Related Disease Association (AARDA) reports that about 1 in 5 Americans suffer from autoimmune disease. They estimate that 75% of those affected are women. They also claim that there are over 80 known autoimmune diseases.

Unfortunately, despite its prevalence, autoimmune disease gets little attention. This is because, unlike cancer, the more than 80 autoimmune diseases are thought of individually rather than collectively. Research and research monies have been inadequate and without focus.

Consequently, the treatments for autoimmune disease have primarily targeted symptoms rather than the underlying cause. Standard treatments for autoimmune diseases include painkillers and immuno-suppressants. These types of treatments have not and will not provide a cure for a single autoimmune disease.

Oral Tolerance

A new method of treating autoimmune disease is called oral tolerance. This method does target the underlying cause of the disease.

Oral tolerance developed from the observation that foreign food proteins (or peptides) which reach the body through the intestines do not normally elicit an immune response.

Thus, if the body tissue under attack in an autoimmune disease could be introduced orally, perhaps the autoimmune response could similarly be suppressed. In other words, orally fed self-antigen could selectively decrease an autoimmune response.

A number of patents have been issued regarding oral tolerance in autoimmune disease. Weiner et al. (U.S. Pat. No. 5,643,868) and Weiner et al. (U.S. Pat. No. 5,763,396) describe methods of treating type 1 diabetes by the oral administration of insulin. Weiner et al. (U.S. Pat. No. 5,720,955), Weiner and Hafler (U.S. Pat. No. 5,733,547), Weiner et al. (U.S. Pat. No. 5,783,188) and Trentham et al. (U.S. Pat. No. 5,399,347) describe methods for treating autoimmune arthritis by the oral administration of soluble collagen, type I or type III collagen, collagen peptide fragments containing repeating sequences, and type II collagen, respectively.

To date, no oral tolerance patents have focused on orally introducing an entire platelet in ITP. Freedman et al. (50) recommended tests of oral immune tolerance using allogeneic platelets, cultured autologous platelets or purified GPIIb-IIIa (platelet membrane glycoprotein) in ITP. This recommendation was an example of a method that is obvious to try, but not obvious to do.

### OBJECTS AND ADVANTAGES

Accordingly, several objects and advantages of my invention are:

(a) to provide a method for treating ITP,

(b) to provide a method that specifically targets the cause of an autoimmune disease,

(c) to provide a method in which enough auto-antigenic material can be manufactured to affect the autoimmune disease, and

(c) to provide a treatment that is inexpensive, safe and effective.

### DRAWING FIGURES

FIG. 1 shows the 4 steps of the preferred embodiment of Oral Tolerance Using Allogeneic Platelets In ITP.

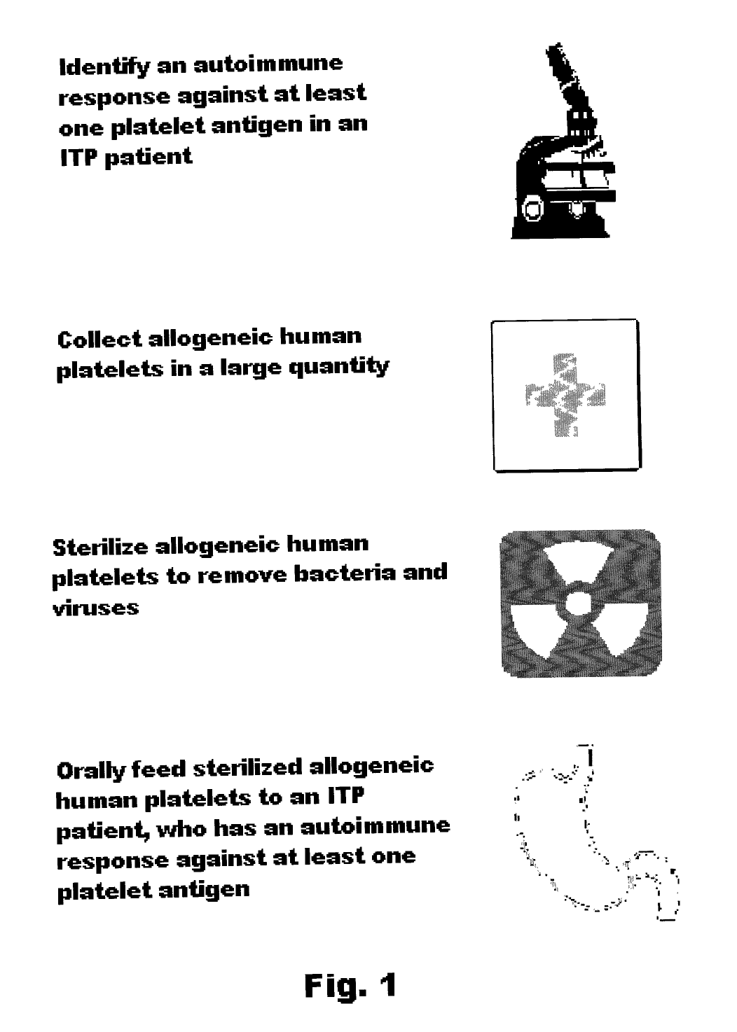

### DESCRIPTION—FIG. 1

FIG. 1

FIG. 1 shows the preferred embodiment of the treatment. The first step in the treatment is the identification an autoimmune response against at least one platelet antigen in an ITP patient.

The second step shown in FIG. 1 is the collection of allogeneic human platelets in a large quantity.

The third step shown in FIG. 1 is the sterilization of allogeneic human platelets to remove bacteria and viruses.

The final step shown in FIG. 1 is the administration of the sterilized allogeneic human platelets to an ITP patient, who has an autoimmune response against at least one platelet antigen. It is thought that the intestines can present antigen to lymphocytes in such a way that causes them to be deactivated or deleted. The fragments must be administered in large enough quantity and for a long enough duration so as to deactivate or destroy enough lymphocytes so as to bring about a decrease in the number of auto-antibodies directed against self tissue.

Preferred Embodiment

ITP

Freedman et al. (25) have described the history of ITP from the first record of symptoms in the 5^{th} century, to research over the last 10 to 15 years. They report that the discoveries made in 1951 were of particular importance. In this year, they note that ITP was induced through plasma transfusion (26), the mechanism of transfer was identified as an anti-platelet antibody (27) and corticosteroids were first used as a treatment (28).

In other words, it was discovered in 1951 that ITP is an autoimmune disease. As a result, idiopathic thrombocytopenic purpura has become synonymous with immune (or immune mediated) thrombocytopenic purpura.

Essentially, the discoveries in 1951 laid the groundwork for our current knowledge of ITP. This knowledge is that ITP is the result of B-cell produced auto-antibodies which are directed against glycoproteins on the platelet cell wall.

Freedman et al. (25) reported another discovery in 1951 that should be noted here. They said that important clinical distinctions were made between acute and chronic ITP (29). This discovery has led some to believe that the underlying autoimmune mechanism in acute and chronic ITP may be different.

There is, however, little evidence that this is the case. Most recently, Semple et al. (30) attempted to correlate differences in serum cytokine levels in patients with acute and chronic forms of the disease with T-cell function. Although they found significantly higher levels of IL-2 in patients with chronic forms of the disease, they were unable to determine if this was a result of the cause of the disease or just a symptom.

Children experience a larger percentage of the acute form of the disease while adults experience a larger percentage of the chronic form of the disease. If ITP were proven to be a T-cell mediated disease, this observation would be logical. T-cell activation or deactivation occurs in the thymus. It is known that the thymus atrophies with age (70 g in infants to only 3 g in the elderly (31)). As a result, it is probably more difficult to deactivate T-cells that have suddenly been directed against self as the body matures.

In short, the clinical labels of acute and chronic ITP should not suggest multiple causes until sufficient evidence is found.

Although 1951 was a watershed year for information about ITP, little of comparable significance has happened in the 47 years since then. Semple and Freedman (32) reported that that a number of groups extensively studied anti-platelet antibodies, and these studies were reviewed by a number of people (33-35). Hou et al. (36) and Wadenvik et al. (37) identified the platelet membrane glycoproteins which act as autoantigens in ITP. Semple and Freedman (32), Semple et al. (30), and Semple (38) have provided evidence that ITP is a T-cell mediated disease.

Since little has been done to extend our knowledge of ITP, very few new treatments have been developed. The standard treatments for ITP include corticosteroids first used in the 1951 (25) and splenectomy first used in 1916 (25). Newer treatments have been borrowed from other diseases or conditions. These include immune globulins, hormones, chemotherapies and other immuno-suppressants.

To date, there is no treatment capable of reversing the disease process or providing sustained reversal of the illness. All current treatments are unsatisfactory because of numerous incapacitating side effects, including death.

Oral Tolerance

There are numerous review articles in the literature regarding the use of oral tolerance in autoimmunity (20, 39-45). These articles describe how the theory of oral tolerance developed from early 20th century experimentation. Most highlight what is currently know about gut-associated immunity. They also suggest that multiple and dose dependent mechanisms are at work in oral tolerance. Finally, many of these articles list the results of animal and human experimentation with oral tolerance.

Oral tolerance developed from the observation that foreign food proteins or peptides which reach the body through the intestines do not normally elicit an immune response. Following this observation various animal experiments were conducted. Typically, animals fed an oral antigen were observed to have developed an insensitivity to that antigen.

Most reviewers of the oral tolerance literature have linked gut-associated immunity to T-cells. Garside and Mowat (44) remarked that although B-cells can be tolerized, T-cells appear to be the most important component. They cited a study (46), which shows the tolerance of B-cells to be less efficient than the tolerance of T-cells.

Most reviewers also agree that there appear to be three mechanisms at work in oral tolerance, and that the mechanism selected is related to the dosage of the oral antigen.

These mechanisms are active suppression, clonal anergy and clonal deletion. Fowler and Weiner (43) reported that active suppression results from low dosages of the oral antigen. They defined active suppression as the induction of antigen-specific cells that suppress the activity of other immune cells by secretion of antiinflammatory cytokines.

Fowler and Weiner (43) suggested that clonal deletion and clonal anergy result from high dosages of oral antigen. They defined clonal anergy as cellular unresponsiveness brought on by the high occupancy of T-cell receptors. This, they claimed, leads to a lack of IL-2 secretion, decreased expression of IL-2 receptors and decreased cell proliferation.

Fowler and Weiner (43) defined clonal deletion as the mechanism bringing about the programmed cell death of T-cells. They suggest that high antigen levels drive specific T-cells into apoptosis, which leads to programmed cell death.

Although Fowler and Weiner (43) described the mechanisms of oral tolerance as dose dependent, they also pointed out that they are not mutually exclusive. In other words, all three mechanisms may be occurring. The dosage, however, determines the mechanism that is favored.

In addition to dosage, frequency of treatment is also a factor. Active suppression appears to require frequent treatments on a repetitive basis (47). In other words, tolerance due to active suppression will end if the treatments are stopped. In contrast, clonal anergy or deletion can result from an infrequent feeding schedule and can be sustained after the treatment is discontinued (47).

A major stumbling block in using oral tolerance to treat autoimmune disease was thought to be the inability to identify the exact autoantigen (39), the possibility of multiple autoantigens (39) and the difficulty in extracting the exact tissue under attack. It was, however, determined that in active suppression regulatory cells induced by oral antigens secreted antigen-nonspecific cytokines (39). As a result, it was shown that it was not necessary to orally introduce the exact antigen. This effect was called bystander suppression.

The reviewers of the oral tolerance literature have reported the effectiveness of this treatment in autoimmune diseases induced in animal models. These diseases have included allergic encephalomyclitis (1,2), collagen induce arthritis (3,4), autoimmune uveitis (5,6), myasthenia gravis (7,8), diabetes mellitus (9,10), transplantation organ rejection (11), autoimmune thyroiditis (12, 13) and granulomatous arthritis (17, 18). Studies in humans have been less successful and have focused on multiple sclerosis (15,16), rheumatoid arthritis (17,18,47) and uveitis (19).

ITP and Oral Tolerance

Although Garside and Mowat (44) found that both B and T-cells have been reported susceptible to oral tolerance, the major focus of oral tolerance research has been T-cell mediated immunity.

The role of T-cell mediated immunity in ITP was largely unknown for many years. McMillan (48) reported that studies in this area were sparse and inconclusive. A number of recent articles (30,32,38,49), however, have shed more light on this area and made a strong case for the role of a T-cell mediated response in ITP.

A possible linkage between ITP and oral tolerance can be found studies describing IL-2 levels in these processes. Semple et al. (30) recorded elevated IL-2 levels in patients with chronic ITP. Fowler and Weiner (43) suggested that IL-2 levels are decreased in high dose oral tolerance that produces clonal anergy or deletion. Because of this linkage, ITP appears to be an excellent candidate for an oral tolerance study.

To date the effectiveness of oral tolerance in human trials has been inconclusive (15-19,47). A human trial of oral tolerance in ITP has two large advantages over all previous trials, however. First of all, the tissue (platelets containing the autoantigen is readily accessible in ITP. This means that the exact antigen can be obtained and introduced orally. Unlike previous trials there is no reliance on bystander suppression, which means that clonal anergy or deletion mechanisms of oral tolerance can be targeted. Secondly, the effectiveness of an oral tolerance treatment in ITP can be objectively assessed. In previous human studies the success of the treatment was judged using subjective measures of improvement in symptoms. A simple blood test will give a completely objective measurement if the treatment's effectiveness in ITP.

Preliminary Studies/Progress Report (Autologous Platelet Oral Tolerance in ITP)

The inventor and Drs. William Bell and Karen King (of Johns Hopkins Hospital) conducted a preliminary study of oral tolerance in ITP using autologous platelets. The subject of this study was a 35 year-old male patient who maintained a count of 17,000 without medication. Two rounds of platelet pheresis and feeding were attempted.

In the first round the patient presented with a count of 17,000 on Jun. 19, 1999. A 5-day course of dexamethasone was begun on Jun. 20, 1999 (20 mg, 20 mg, 20 mg, 20 mg and 10 mg). On the third day of dexamethasone, Jun. 22, 1999, plateletpheresis was performed on the patient. The platelet count before pheresis was 162,000. The post-pheresis count was 85,000. Approximately, 15 grams of wet platelets were collected.

One day after the course of dexamethasone, Jun. 25, 1999, plateletpheresis was repeated. The pre-pheresis count was 311,000 and the post-pheresis count was 148,000. Approximately, 23 grams of wet platelets were collected.

The collected platelets were consumed within a week of their collection in order to preserve their chemical integrity. The first feeding took place on Jun. 28, 1999. 15 grams of wet platelets were consumed on an empty stomach. Saline was used to help swallow the platelets. Food was not taken until at least 2 hours after the feeding. The second feeding occurred on Jun. 29, 1999. Approximately, 23 grams of wet platelets were ingested on an empty stomach using saline. Food again was not taken until at least 2 hours after the feeding.

The platelet count before the first feeding was 108,000. The platelet count after the second feeding declined from 60,000 to 14,000 in 8 days. Over the next 7 days it rose again to 87,000. From there it stabilized to an average count of about 55,000 over 6 weeks.

The platelet counts for the first round of plateletpheresis and platelet feeding is shown in FIG. A.

[image]

[image]

[image]

round of pheresis and feeding. Because of the use of dexamethasone to raise the count for pheresis it was not possible to determine if the higher count after feeding was due to the dexamethasone or due to the feeding. The second round, without the use of dexamethasone, was meant to resolve this issue. Unfortunately, the lower count produced a significantly lower collection of platelets. Mokhtarian F, et al. (53), has also reported detrimental effects of dexamethasone on oral tolerance.

2. Autologous platelets are difficult to obtain and store in large quantities. Without medications to increase the pre-pheresis count, many rounds of plateletpheresis are required to obtain sufficient amounts of platelets. This is very difficult for patients. Also, because of limitations on the storage of platelets, the platelets that were collected in this study had to be fed within a week of collection. This meant that is was not possible to spread the feeding out of a number of days as it was done in most animal studies.

3. Although difficult, repeat plateletpheresis is possible in ITP patients. The rapid recovery of the platelet count after pheresis in round 2 showed that is possible to safely perform repeat plateletpheresis on ITP patients.

In addition to the work of the inventor, Dr. Bell and Dr. King, Wadenvik H, et al. (37) have suggested that immune tolerance be explored as a possible treatment in ITP. Also, Freedman et al. (50) recommended tests of oral immune tolerance using allogeneic platelets, cultured autologous platelets or purified GPIIb-IIIa (platelet membrane glycoprotein).

Dosage

According to Whitacre et al., (20), multiple feedings were superior to single feedings and large dosages were preferable to small dosages. They fed a total of 20 mg of protein over four days to rats in their multiple sclerosis studies. If a rat weighs 0.3 kg and the average human weighs 70 kg, at least 4.67 grams of protein would be needed for each patient in a human study.

Not all platelet protein is antigenic in ITP. Assuming that 5% is antigenic, at least 93.4 grams of total platelet protein would be required for each patient. According to Bithell (52) 1 gram of wet platelets equals 119 milligrams of protein, which is equivalent to 0.78×10(11) platelets. Therefore, 783 grams of wet platelets and 613×10(11)platelets are needed for each patient.

Assuming that a single platelet donation contains 5.5×10(11)platelets, approximately 112 donations would be required for feeding one patient.

In the only successful demonstration of oral tolerance in humans Husby et al., (53), fed a total of 0.5 g antigen over 19 days. There were 10 feedings on days 1 to 5 and 15 to 19.

Using this protocol, approximately 11 platelet donations would be fed during each feeding.

Research Design/Methods

Ten patients with chronic ITP will be recruited from the clinical practice. Each should have a count without medication between 10,000 and 80,000. All ITP medications will be discontinued.

Expired human platelet donations will be obtained for each patient from the Red Cross. The platelets will be spun down to pellet form and the pellet will be frozen.

When 112 donations for a patient have been obtained, these donations will be sterilized to remove any harmful bacteria or viruses. Exposing the donations to 10 kGY of gamma radiation will perform sterilization. This level of radiation will destroy bacteria and viruses without affecting the platelet proteins (54).

A patient will be fed the sterilized pellets over 19 days. Feedings will occur on days 1 to 5 and 15 to 19 as Husby et al., (53), have outlined. A patient will be fed 11 frozen donations of platelets in pellet form on an empty stomach in each feeding. The platelets may be fed with frozen yogurt. Nothing else will be eaten for at least two hours after the feeding.

A platelet count and autoantibody titer will be obtained before the first feeding and a platelet count will be obtained after the last feeding. Platelet counts will then be obtained every two weeks in the first month after treatment and monthly for five additional months. An autoantibody titer will be obtained six months after the feeding. Success will be defined as a 50% increase in the platelet count and a decline in the autoantibody titer six months after the feeding.

Risks

There are two major risks involved in this study. First of all, it is possible that the therapy could cause a rapid decline in the platelet count. This may be the result of the therapy or the normal progression of the count for a particular patient. No one has reported that orally introduced antigens worsen autoimmune diseases in humans. However, there have been a number of reports of oral antigens causing autoimmune disease in animal models (21,22,23,24). In any event, careful monitoring of the patient in the study should prevent any problems caused by a rapidly declining platelet count.

The second major risk involves the transmission of disease from allogeneic platelets prepared for consumption. Expired human platelets have already gone through an extensive screening process. The additional step of irradiating the platelets before feeding will also be taken in this study.

### REFERENCES

1. Higgins P J, Weiner H L, Suppression of experimental autoimmune encephlomyelitis by oral administration of myelin basic protein and its fragments. J Immunol Jan. 15, 1988; 140 (2):440-445.

2. Kelly K A, Whitacre C C, Oral tolerance in EAE: Reversal of tolerance by T helper cell cytokines. Neuroimmunol. May 1996; 66(1-2):77-84

3. Thompson H S, Harper N, Bevan D J, Staines N A, Suppression of collagen induced arthritis by the adminstration of type II collagen: changes in immune and arthritic responses mediated by active peripheral suppression. Autoimmunity 1993; 16(3):189-199.

4. Nagler-Anderson C, Bober L A, Robinson M E, Siskind G W, Thorbecke G J. Suppresion of type II collagen-induced arthritis by intragastric administration of soluble type II collagen. Proc Natl Acad Sci USA October 1986; 83 (19): 7443-7446.

5. Gregerson D S, Obritsch W F, Donoso L A, Oral tolerance in experimental autoimmune uveoretinitis. Distinct mechanisms of resistance are induced by low dose vs high dose feeding protocols. J Immunol Nov. 15, 1993; 151(10):5751-61

6. Nussenblatt R B, Caspi R R, Mahdi R, Chan C C, Roberge F, Lider O, Weiner H L, Inhibition of S-antigen induced experimental autoimmune uveoretinitis by oral induction of tolerance with S-antigen. J Immunol. Mar. 1, 1990;144(5):1689-95.

7. Wang Z Y, Huang J,. Olsson T, He B, Link H, B cell responses to acetylcholine receptor in rats orally tolerized against experimental autoimmune myasthenia gravis. J Neurol Sci. February 1995;128(2):167-74.

8. Okumura S. McIntosh K, Drachman D B, Oral administration of acetylcholine receptor: effects on experimental myasthenia gravis. Ann Neurol. November 1994;36(5):704-13.

9. Hancock W W, Polanski M, Zhang J, Blogg N, Weiner H L, Suppression of insulitis in non-obese diabetic (NOD) mice by oral insulin administration is associated with selective expression of interleukin-4 and -10, transforming growth factor-beta, and prostaglandin-E. Am J Pathol. November 1995;147(5):1193-9.

10. von Herrath M G, Dyrberg T, Oldstone M B, Oral insulin treatment suppresses virus-induced antigen-specific destruction of beta cells and prevents autoimmune diabetes in transgenic mice. J Clin Invest. Sep. 15, 1996;98(6):1324-31.

11. Sayegh M H, Zhang Z J, Hancock W W, Kwok C A, Carpenter C B, Weiner H L, Down-regulation of the immune response to histocompatibility antigens and prevention of sensitization by skin allografts by orally administered alloantigen. Transplantation. January 1992;53(1):163-6.

12. Guimaraes V C, Quintans J, Fisfalen M E, Straus F H, Wilhelm K, Medeiros-Neto A, DeGroot L J, Suppression of development of experimental autoimmune thyroiditis by oral administration of thyroglobulin. Endocrinology. August 1995;136(8): 3353-9.

13. Peterson K E, Braley-Mullen H, Suppression of murine experimental autoimmune thyroiditis by oral administration of porcine thyroglobulin. Cell Immunol. November 1995;166(1):123-30.

14. Neurath M F Fuss I, Kelsall B L, Presky D H, Waegell W, Strober W, Experimental granulomatous colitis in mice is abrogated by induction of TGF-beta-mediated oral tolerance. J Exp Med. Jun. 1, 1996;183(6):2605-16.

15. Kent S C, Fukaura H, Pietrusewicz M J, Khoury S J, Weiner H L, Hafler D A, Oral administration of myelin induces antigen-specific TGF-beta 1-secreting T cells in multiple sclerosis patients. Ann N Y Acad Sci. Apr. 5, 1997;815:412-22.

16. Weiner H L, Mackin G A, Matsui M, Orav E J, Khoury S J, Dawson D M, Hafler D A, Double-blind pilot trial of oral tolerization with myelin antigens in multiple sclerosis. Science. Feb. 26, 1993;259(5099): 1321-4.

17. Sieper J, Kary S, Sorensen H, Alten R, Eggens U, Huge W, Hiepe F, Kuhne A, Listing J, Ulbrich N, Braun J, Zink A, Mitchison N A, Oral type II collagen treatment in early rheumatoid arthritis. A double-blind, placebo-controlled, randomized trial. Arthritis Rheum. January 1996;39(1):41-51.

18. Barnett M L, Combitchi D, Trentham D E, A pilot trial of oral type II collagen in the treatment of juvenile rheumatoid arthritis. Arthritis Rheum. April 1996;39(4):623-8.

19. Nussenblatt R B, Gery I, Weiner H L, Ferris F L, Shiloach J, Remaley N, Perry C, Caspi R R, Hafler D A, Foster C S, Whitcup S M, Treatment of uveitis by oral administration of retinal antigens: results of a phase I/II randomized masked trial. Am J Ophthalmol. May 1997;123(5):583-92.

20. Whitacre C C, Gienapp I E, Meyer A, Cox K L, Javed N, Treatment of autoimmune disease by oral tolerance to autoantigens. Clin Immunol Immunopathol. September 1996;80(3 Pt 2):S31-9.

21. Meyer A L, Benson J M, Gienapp I E, Cox K L, Whitacre C C, Suppression of murine chronic relapsing experimental autoimmune encephalomyelitis by the oral administration of myelin basic protein. J Immunol. Nov. 1, 1996;157(9):4230-8.

22. Blanas E. Carbone F R, Allison J, Miller J F, Heath W R, Induction of autoimmune diabetes by oral administration of autoantigen. Science. Dec. 6, 1996;274(5293):1707-9.

23. Terato K, Ye X J, Miyahara H, Cremer M A, Griffiths M M, Induction by chronic autoimmune arthritis in DBA/1 mice by oral administration of type II collagen and Escherichia coli lipopolysaccharide. Br J Rheumatol. September 1996;35(9):828-38.

24. Miller A, Lider O, Abramsky O, Weiner H L, Orally administered myelin basic protein in neonates primes for immune responses and enhances experimental autoimmune encephalomyelitis in adult animals. Eur J Immunol. May 1994;24(5): 1026-32.

25. Freedman J, et al. Idiopathic thrombocytopenic purpura (ITP): a historical odyssey. Acta Paediatr Suppl. June 1998; 424: 3-6.

26. Harrington W J, et al. Demonstration of a thrombocytopenic factor in the blood of patients with thrombocytopenic purpura. J Lab Clin Med 1951; 38: 1-10

27. Evans R S, et al. Primary thrombocytopenic and acquired hemolytic anemia: evidence for a common etiology. Arch Intern Med 1951; 87 48-65

28. Wintrobe M M, et al. Effect of corticotropin and cortisone on the blood in various disorders in man. Arch Intern Med 1951; 88: 310-36

29. Hirsh E O and Dameshek W., ‘Idiopathic’ thrombocytopenia. Arch Intern Med 1951; 88: 701-28

30. Semple J W et al. Differences in serum cytokine levels in acute and chronic autoimmune thrombocytopenic purpura: relationship to platelet phenotype and antiplatelet T-cell reactivity. Blood. May 15, 1996; 87(10): 4245-4254.

31. Kubis J. Immunology, W. H. Freeman and Company, New York, 1994, p. 71

32. Semple J W and J Freedman Abnormal cellular immune mechanisms associated with autoimmune thrombocytopenia. Transfus Med Rev. Oct. 1, 1995; 9(4): 327-338

33. Kiefel V et al. Serological, biochemical, and molecular aspects of platelet autoantigens. Semin Hematol 29: 26-33, 1992

34. Waters A H Autoimmune thrombocytopenia: Clinical Aspects. Semin Hematol 29: 18-25, 1992

35. Kunicki T J and P J Newman The molecular immunology of human platelet proteins. Blood 80:1386-1404, 1992

36. Hou M, et al. Antibodies against platelet GPIb/IX, GPIIb/IIIa, and other platelet antigens in chronic idiopathic thrombocytopenic purpura. Eur J Haematol. Nov. 1, 1995; 55(5): 307-314.

37. Wadenvik H, et al. Platelet proteins as autoantibody targets in idiopathic thrombocytopenic purpura. Acta Paediatr Suppl. June 1998; 424: 26-36.

38. Semple J W. Immunobiology of T helper cells and antigen-presenting cells in autoimmune thrombocytopenic purpura. Acta Paediatr Suppl. June 1998; 424: 41-45

39. Weiner H L. Oral tolerance: immune mechanisms and treatment of autoimmune diseases. Immunol Today. Jul. 1, 1997; 18(7): 335-343

40. Weiner H L. Oral tolerance for the treatment of autoimmune diseases. Annu Rev Med. Jan. 1, 1997; 48: 341-351

42. Weiner H L, et al. Oral tolerance: mechanisms and applications. Introduction. Ann N Y Acad Sci. Feb. 13, 1996; 778: XIII-XVIII

43. Fowler E and H L Weiner Oral tolerance: elucidation of mechanisms and application to treatment of autoimmune diseases. Biopolymers. Jan. 1, 1997; 43(4): 323-335

44. Garside P and A M Mowat Mechanisms of oral tolerance. Crit Rev Immunol. Jan. 1, 1997; 17(2): 119-137

45. Brandtzaeg P. The human intestinal immune system: basic cellular and humoral mechanisms. Baillieres Clin Rheumatol. Feb. 1, 1996; 10(1): 1-24

46. Vives J et al. Immunologic unresponsiveness after gastric administration of human gamma-globulin: antigen requirements and cellular parameters, J Immunol, 125, 1811, 1980

47. Trentham D E Oral tolerization as a treatment of rheumatoid arthritis. Rheum Dis Clin North Am. August 1998; 24(3): 525-536

48. McMillan R. Chronic idiopathic thrombocytopenic purpura. N Engl J Med. May 7, 1981; 304(19): 1135-1147

49. Semple J W, et al. Cellular immune mechanisms in chronic autoimmune thrombocytopenic purpura (ATP). Autoimmunity. Jan. 1, 1992; 13(4): 311-319

50. Freedman J, et al. Directions for research in autoimmune thrombocytopenic purpura. Acta Paediatr Suppl. June 1998; 424: 82-84

51. Rock G, Tittley P, Sternbach M, Buskard N, Schroeder M, Department of Medicine, University of Ottawa, Canada, Repeat plateletpheresis: the effects on the donor and the yield., Vox Sang 1992;63(2):102-106

52. Wintrobe's Clinical Hematology, Ninth Edition, vol 1, Chapter 17, Platelets and Megakaryocytes, Thomas Bithell

53. Husby S. et al. Oral tolerance in humans: T cell but not B cell tolerance to a soluble protein antigen. Adv Exp Med Biol. 1995;371B:1225-8.

54. Facts about food irradiation, International Consultative Group on Food Irradiation, 1999.

Summary, Ramifications, and Scope

Accordingly, the reader will see that the invention of Oral Tolerance Using Allogeneic Platelets in ITP will finally allow the underlying cause of these diseases to be affected:

Additional advantages of this invention are:

little or no side-effects;

the ability to target the treatment to the patient; and

the possibility of a sustained response without further medication.

Although the description above contains many specificities, these should not be construed as limiting the scope of the invention but as merely providing illustrations of some of the presently preferred embodiments of this invention.

Thus the scope of the invention should be determined by the appended claims and their legal equivalence, rather than the examples given.

I claim:

1. A method for treating Immune Thrombocytopenic Purpura comprising:
(a) identification of an autoimmune response against at least one platelet antigen in an ITP patient,
(b) collection of allogeneic human platelets in a large quantity,
(c) sterilization of said allogeneic human platelets to remove harmful bacteria and viruses, and
(d) oral feeding of said sterilized allogeneic human platelets to said ITP patient in large enough quantity and for long enough duration so as to bring about a decrease in the autoimmune response.
